(19) United States
(12) Patent Application Publication
(10) Pub. No.: US 2005/0267570 A1
(43) Pub. Date: Dec. 1, 2005

(54) Endovascular occlusion devices and methods of use

(76) Inventor: John H. Shadduck, Menlo Park, CA (US)

Correspondence Address: John H. Shadduck, #B23, 350 Sharon Park Drive, Menlo Park, CA, 94025, US

(21) Appl. No.: 11/140,421

(22) Filed: May 27, 2005

Related U.S. Application Data
(60) Provisional application No. 60/575,081, filed on May 27, 2004.

(51) Int. Cl. A61F 2/06

(52) U.S. Cl. 623/1.44

(57) ABSTRACT

An implantable stent-like device for treating and occluding arteriovascular malformations (AVMs), fistulas, varicose veins or the like. More particularly, this invention relates to an implant body that includes an open-cell shape-transformable polymer structure that provides stress-free means for occluding an AVM without applying additional pressures an any distended walls of the AVM. In one embodiment, the shape-transformable polymer is a shape memory polymer implant body that self-deploys from a temporary shape to a memory shape. In another embodiment, the shape memory polymer structure is capable of a temporary compacted shape for carrying about the struts of an expandable stent for self-deployment to occlude an aneurysm.

### CROSS-REFERENCE TO RELATED APPLICATION

This application claims benefit of Provisional U.S. Patent Application Ser. No. 60/575,081 filed May 27, 2004 titled Endovascular Occlusion Devices and Methods of Fabrication.

### BACKGROUND OF THE INVENTION

1. Field of the Invention

This invention relates to devices for occluding arteriovascular malformations (AVMs), fistulas or blood vessels. More particularly, this invention relates to an implant body that includes a shape-transformable polymeric structure for self-deployment within vasculature, and can be an open-cell shape memory polymer in the form of a microfabricated structure or a foam and also can be carried about a skeletal stent to provide stress-free means for occluding an AVM without applying additional pressures to the distended walls of an AVM.

2. Description of the Related Art

Numerous vascular disorders, as well as non-vascular disorders, are treated by occluding blood flow through a region of the patient's vasculature. For example, aneurysms, fistulas, varicose veins and the like are treated with vessel occluding devices. Tumors and the like are also treated with endovascular embolic elements to terminate blood flow. Several procedures are described below.

An intracranial aneurysm is a localized distension or dilation of an artery due to a weakening of the vessel wall. In a typical example, a berry aneurysm is a small bulging, quasi-spherical distension of an artery that occurs in the cerebral vasculature. The distension of the vessel wall is referred to as an aneurysm sac, and may result from congenital defects or from preexisting conditions such as hypertensive vascular disease and atherosclerosis, or from head trauma. Up to 2% to 5% of the U.S. population is believed to harbor an intracranial aneurysm. It is has been reported that there are between 25,000 and 30,000 annual intracranial aneurysm ruptures in North America, with a resultant combined morbidity and mortality rate of about 50%. (See Weir B., Intracranial aneurysms and subarachnoid hemorrhage: an overview, in Wilkins R. H., Ed. Neurosurgery, New York: McGraw-Hill, Vol. 2, pp 1308-1329 (1985)).

Rupture of a cerebral aneurysm is dangerous and typically results in bleeding in the brain or in the area surrounding the brain, leading to an intracranial hematoma. Other conditions following rupture include hydrocephalus (excessive accumulation of cerebrospinal fluid) and vasospasm (spasm of the blood vessels).

Several methods of treating intracranial aneurysms are known including open surgeries and endovascular procedures. In an open craniotomy, a clip is placed at the base of the aneurysm. Long-term studies have established typical morbidity, mortality, and recurrence rates.

The least invasive approach for treating intracranial aneurysms is an endovascular method—which consists of a reconstructive procedure in which the parent vessel is preserved. Luessenhop developed the first catheter-based treatment of an intracranial aneurysm (see Luessenhop A. J., Velasquez A. C., Observations on the tolerance of intracranial arteries to catheterization, J. Neurosurg. 21:85-91 (1964)). At that time, technology was not yet developed for successful outcomes. Serbinenko and others deployed latex balloons in intracranial aneurysms (see Serbinenko, F. A., Balloon catheterization and occlusion of major cerebral vessels, J. Neurosurg. 41:125-145 (1974)) with mixed results.

More recently, Guglielmi and colleagues succeeded in developing microcatheter-based systems (GDC or Guglielmi detachable coil systems) that deliver very soft platinum microcoils into an aneurysm to mechanically occlude the aneurysm sac. After the position of the microcoil is believed to be stable within the aneurysm sac, the coil is detached from the guidewire by means of an electrolytic detachment mechanism and permanently deployed in the aneurysm. If coil placement is unstable, the coil can be withdrawn, re-positioned or changed-out to a coil having different dimensions. Several coils are often packed within an aneurysm sac. Various types of such embolic coils are disclosed in the following U.S. patents by Guglielmi and others: Nos. 5,122,136; 5,354,295; 5,843,118; 5,403,194; 5,964,797; 5,935,145; 5,976,162 and 6,001,092.

Microcatheter technology has developed to permit very precise intravascular navigation, with trackable, flexible, and pushable microcatheters that typically allow safe engagement of the lumen of the aneurysm. However, while the practice of implanting embolic coils has advanced technologically, there still are drawbacks in the use of GDC-type coils. One complication following embolic coil implantation is subsequent recanalization and thromobembolitic events. These conditions are somewhat related, and typically occur when the deployed coil(s) do not sufficiently mechanically occlude the volume of the aneurysm sac to cause complete occlusion. Recanalization, or renewed blood flow through the aneurysm sac, can cause expansion of the sac or migration of emboli from the aneurysm. Recanalization can occur after an implantation of a GDC coil if the coil does not form a sufficiently complete embolus in the targeted aneurysm. After the initial intervention, the body's response to the foreign material within the vasculature causes platelet activation etc., resulting in occlusive material to build up about the embolic coil. After an extended period of time, the build-up of occlusive material about the foreign body will cease. If spaces between the coils and occlusive material are too large, blood flow can course through these spaces thus recanalizing a portion of the thin wall sac. The blood flow also can carry emboli from the occlusive material downstream resulting in serious complications.

Alternative treatments include endovascular occlusion of the aneurysm with a liquid polymer that can polymerize and harden rapidly after being deployed to occlude the aneurysm. Wide neck aneurysms make it difficult to maintain embolic or occlusive materials within the aneurysmal sac—particularly liquid embolic materials. Such embolic materials can dislocate to the parent vessel and poses a high risk of occluding the parent vessel.

Another approach in the prior art is to provide an aneurysm liner of a woven or braided polymeric material such as polypropylene, polyester, urethane, teflon, etc. These mesh materials are difficult to use in treating larger aneurysms, since the materials cannot be compacted into a small diameter catheter.

Any method of endovascular occlusion with packing materials risks overfilling the sac and also the risk of agent migration into the parent vessel. Any overfill of the sac also will cause additional unwanted pressure within the aneurysm.

Another past method for occluding aneurysm sac used an elastic, expandable balloon member or liner that was introduced into the aneurysm and thereafter detached from the catheter. Such balloon implants are not likely to conform to the contours of an aneurysm and thus allow blood canalization about the balloon surface. A balloon also can cause undesired additional pressure on the aneurysm wall if oversized. The deployment and implantation of a balloon that carries stresses that may be released in uncontrollable directions is highly undesirable. Such balloon treatments have been largely abandoned.

Further, there are some aneurysm types that cannot be treated effectively with an endovascular approach. In such cases, the treatment options then may be limited to direct surgical intervention—which can be highly risky for medically compromised patients, and for patient that have difficult-to-access aneurysms (e.g., defects in the posterior circulation region).

The first type of intracranial aneurysm that cannot be treated effectively via an endovascular approach is a wide-neck aneurysm. In many aneurysms, the shape of the aneurysm sac is shape like a bowler's hat, for example, in which the neck/dome ratio is about 1:1. For the best chance of success in using an embolic coil, an intracranial aneurysm should have a narrow neck that allows the coils to be contained inside the aneurysmal sac. Such containment means that migration of the coil is less likely, and the possibility of thromboembolic events is reduced. To promote coil stability in wide-neck aneurysms, surgeons have attempted to temporarily reduce the size of the aneurysm neck by dilating a non-detachable balloon during coil deployment thereby allowing the coils to engage the walls of the sac while the neck is blocked. Another type of aneurysm that proves difficult to occlude with embolic coils is a fusiform aneurysm that bulges a large portion of the vessel lumen. Yet another type of aneurysm that responds poorly to endosaccular coiling is a giant aneurysm. In these cases, the recanalization rates remain high, the risk for thromboembolic phenomena is high, and the mass effect persists which related to the lack of volume reduction over time. The treatment of abdominal aortic aneurysms also would benefit from new implant systems that will better engage the vessel wall and occlude the distended vessel wall.

What is needed, in particular, are vaso-occlusive systems and techniques that are reliable and self-deploying for many types of vascular disorders, for example to occlude varicose veins. In particular, improved systems are needed for endovascular treatment of bifurcation aneurysms, wide-neck aneurysms, fusiform aneurysms and giant aneurysms that can provide acceptable outcomes.

### SUMMARY OF THE INVENTION

The present invention is directed to implants and methods for treating arteriovascular malformations (AVMs), varicose veins and the like. The exemplary embodiments and methods are described in treating cerebral aneurysms, but it should be appreciated that the inventions can be applied to other vascular defects, fistulas, cavities and the like.

Of particular interest, the present invention is adapted for treating all different types of aneurysms that typically present different types of challenges. For example, various embodiments of implants of the invention are adapted for treating wide-neck aneurysms and fusiform aneurysms.

In one preferred embodiment, an exemplary implant of the invention comprises an implant body of an open-cell shape-transformable polymer for absorbing pulsatile effects of blood flow about an aneurysm. In one embodiment, the implant body is microfabricated of a shape memory polymer by soft lithography means to provide a selected open-cell structure, or a gradient in open-cell volume, to insure that the implant will induce rapid formation of thrombus substantially without any packing pressure that would risk distention of an aneurysm sac.

In another preferred embodiment, a shape-transformable polymer structure is coupled to a superelastic nickel titanium alloy stent for use in interventional neuroradiology for rapid deployment from a catheter.

In one aspect of the invention, a shape memory polymer structure is at least partially of a bioabsorbable or bioerodible open-cell polymer.

In another aspect of the invention, the open-cell shape memory polymer structure has a very low structure modulus and is carried about the struts of a stent.

In another aspect of the invention, the open-cell shape memory polymer structure can be temporarily compacted for catheter deployment and expand to a suitable dimension to occupy aneurysms greater that about 10 mm. in diameter.

In another aspect of the invention, a highly elongate open-cell shape memory polymer structure can be inserted into a varicose vein to occlude the vein after expansion to a memory shape.

### BRIEF DESCRIPTION OF THE DRAWINGS

Other objects and advantages of the present invention will be understood by reference to the following detailed description of the invention when considered in combination with the accompanying Figures, in which like reference numerals are used to identify like components throughout this disclosure.

FIG. 1A is a schematic view of an open-cell microfabricated shape memory polymer (SMP) implant body or stent in the treatment of a bifurcation aneurysm, the stent in a deployed configuration with a dashed line indicating its pre-deployed shape.

FIG. 1B is a schematic view of an alterative open-cell microfabricated SMP implant body in a deployed configuration in a wide neck aneurysm after introduction through a skeletal wire stent.

FIG. 1C is an illustration of the use of an alterative open-cell microfabricated SMP implant body in a highly elongated form for occluding a varicose vein.

FIG. 2 is a sectional view of the open-cell shape memory body of FIG. 1 in its stress-free memory shape.

FIG. 3 is a view of the open-cell shape memory body of FIG. 2 in its stressed temporary shape.

FIG. 4A is a greatly enlarged perspective schematic view of the open-cell shape memory elastomeric structure of FIG. 2 and in its stress-free memory configuration.

FIG. 4B is a schematic view of the open-cell elastomeric structure of FIG. 4A being deformed toward a selected stressed configuration.

FIG. 5 is an exploded, sequential view of a method of assembling an open-cell shape memory elastomeric structure with a superelastic nickel-titanium alloy stent, the elastomeric structure in a memory shape and a temporary compacted shape.

FIG. 6 s a view similar to that of FIG. 5 depicting the open-cell shape memory elastomeric structure in a temporary compacted configuration.

FIG. 7 is a view of the assembling stent of FIG. 5 in a collapsed position within a catheter sleeve, the shape memory polymer structure in a temporary compacted shape.

FIG. 8 is a view of the stent and open-cell shape memory elastomeric structure of FIG. 7 deployed in a wide neck aneurysm.

FIG. 9 is a schematic view of a portion of a stent deployed in a wide-neck intracranial aneurysm with local perforators.

FIG. 10 is a view of a portion of a stent deployed in across a wide-neck intracranial aneurysm with local perforators, the SMP being a thin layer.

FIGS. 11 and 12 are schematic views of an alternative stent with a large surface area carrying a shape memory polymer structure for treating a fusiform aneurysm.

FIG. 13 is a schematic view of an alternative bifurcated stent for treating an aortic aneurysm.

FIG. 14 is an exploded view of a thin shape-transformable polymer structure carried about at least one cell of the struts of a stent.

### DESCRIPTION OF PREFERRED EMBODIMENTS OF THE INVENTION

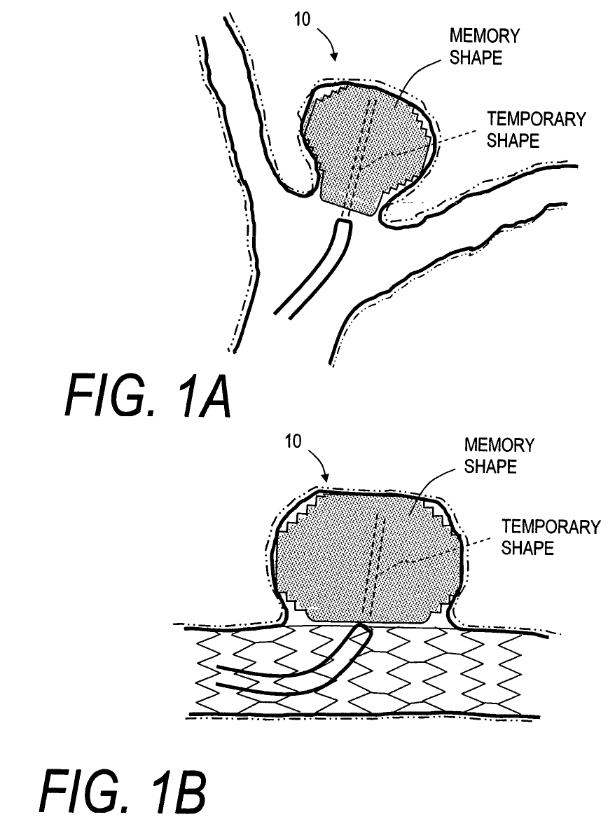

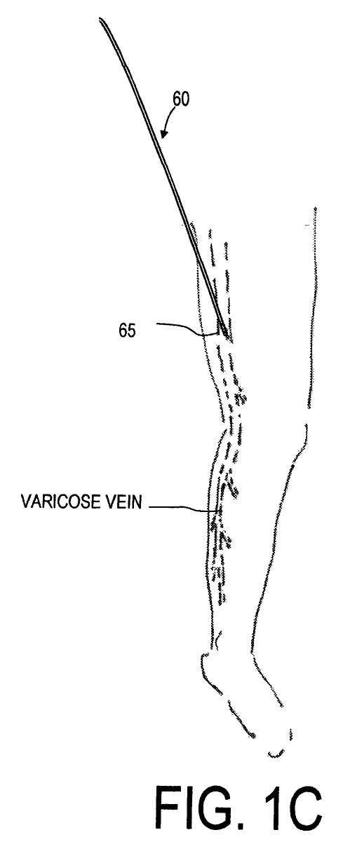

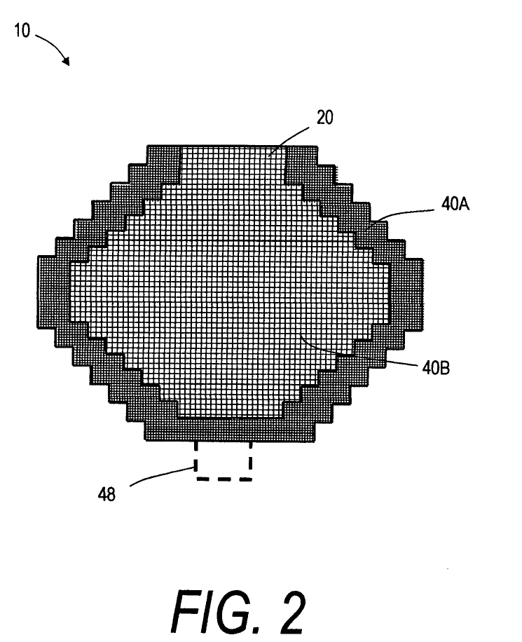

FIG. 1A illustrates the lumen of a common form of intracranial AVM usually described as a bifurcation aneurysm 5 that is often difficult to treat with embolic coils. The implant or stent corresponding to the invention comprises and open-cell or open-volume elastomeric shape memory polymer (SMP) monolith or body 10 that is capable of a “memory” extended or expanded shape as in FIG. 1A and is self-deployable from a “temporary” non-extended or compacted shape (phantom view). FIG. 2 illustrates a sectional view of an exemplary microfabricated elastomeric SMP body 10 in its memory “shape”, and FIG. 3 illustrates the stent 10 in its “temporary” equilibrium compacted shape.

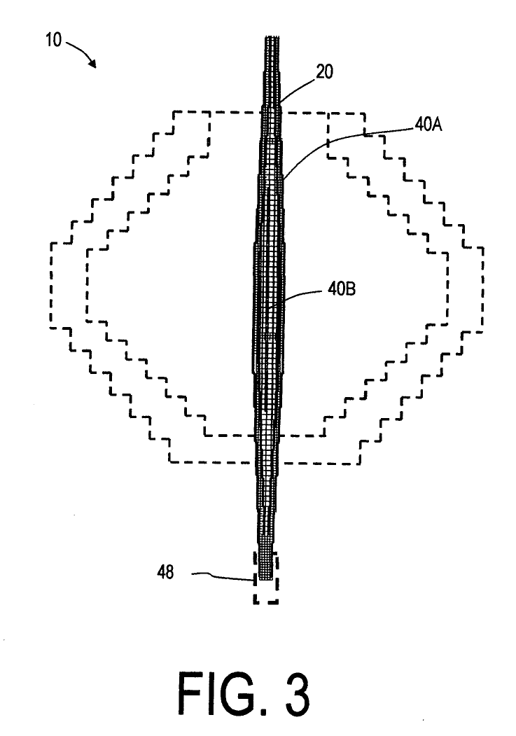

In a preferred embodiment, the stent 10 of FIG. 1A has an open-cell elastomeric structure and is fabricated by soft lithography microfabrication means resulting in a open-cell structure 20 as depicted in FIG. 4A. An alternative method of making open-cell structure is by a polymer foaming process. The open-cell polymer element 20 of FIG. 4A in its memory shape or deployed configuration, among other advantages, is adapted for absorbing pulsatile forces caused by heart beats about the neck of an aneurysm. The open-cell elastomeric element body 20 (FIG. 4A) is further adapted to expand and at least partly occupy an AVM to limit or eliminate blood circulation within an aneurysm sac. Other embodiments can be shaped and adapted to occupy only the neck portion of an aneurysm, which thereafter can cause flow stagnation and natural thrombus formation to entirely occlude an aneurysm sac.

In preferred embodiments, the open-cell dimensions C of the elastomeric structure 20 of FIG. 4A can range from mean (open-cell) cross sections of 1 microns to 500 microns, and more preferably from about 10 microns to 100 microns. A more preferred manner of describing the invention is that the microfabricated shape memory polymer body has an open cell interior volume of at least 50 percent of the volume of the body in a stress free state. More preferably, the microfabricated SMP body has an open cell interior volume of at least 60 percent of the volume of the body in a stress free state, and still more preferably the open cell interior volume is at least 70 percent.

Of particular interest, as depicted in FIG. 4B, the microfabricated open-cell elastomeric structure 20 of FIG. 4A can be designed for controlled deformation of polymer network elements 22a, 22b and 22c that extend in different planes or direction. Such controlled deformation and releasable containment of an ordered open structure in the temporary compacted position allow for optimization of compacting, wherein microfabrication by means of foaming provides less 3D control over compaction of the structure. Further, such ordered open polymer networks allow for control of the direction of expansion forces by the implant when released from its temporary compacted position.

In FIG. 2, the open-cell elastomeric monolith 10 in its memory, repose shape is in a stress-free state. In one preferred embodiment, the structure 10 of FIG. 2 has first and second polymer blocks, 40A and 40B. A surface block 40A comprises of a shape memory polymer (SMP) block that defines a transition characteristic (described below) at which its changes from a higher modulus state to its rubbery modulus that allows SMP block 40A to releaseably constrain interior block 40B in a deformed, compacted state. The interior block can be a SMP or a very low modulus open-cell elastomer. In other words, the SMP block 40A can constrain the entire implant in a deformed shape thereby storing mechanical energy that was expended during deformation of the implant. The SMP composition is fabricated to have a selected memory shape and modulus that is compactable or deformable to a cylindrical shape (FIG. 3) to allow the implant to be carried in the lumen of a catheter for minimally invasive introduction into the aneurysm sac 5. The implant then expands to its memory shape in response to temperature or another selected stimulus when ejected from the catheter.

As illustrated schematically in FIG. 2, the implant 10 has an edge that appears “stepped” which is somewhat exaggerated. Soft lithography microfabrication in successive layers can result in such stepped configurations layer-to-layer. Thus, the implant 10 is capable of the first compacted cross-sectional shape of FIG. 3 and the second expanded cross-sectional shape of FIG. 2 to substantially occupy an aneurysm (FIGS. 1A-1B). The implant 10 of FIGS. 2-3 also is preferably a bioerodible/bioabsorbable polymeric composition that over a period ranging from about 1 to 100 weeks will substantially be eroded or absorbed by the body thus allowing for elimination of any long-term bulk effect in the patient after treatment, which is to be distinguished from packing an aneurysm with metallic embolic coils.

As depicted schematically in FIG. 2, the implant 10 further has release means indicated at 48 which can be any release means known in the art, such as electrolytic joints or other mechanical or sacrificial releases.

It should be appreciated that the implant 10 (FIG. 1A) also could comprise a body of a single SMP composition. In another preferred embodiment, the body can have a gradient in open-cell volume with lesser open volume near the neck region and the surfaces of the implant.

As can be seen in FIG. 1B, the open-cell microfabricated body also be introduced into a wide neck aneurysm in cooperation with a skeletal wire stent as is known in the art.

Referring to FIG. 1C, it can be easily understood that an SMP implant body 60 can be highly elongated with a memory shape having a cross-section ranging between about 4 and 20 mm. for occluding a varicose vein. The implant can be compacted to a temporary shape that is in the range of about 0.5 to 3.0 mm in diameter for introduction in the vessel through and incision 65. The SMP body 60 can be adapted to expand slowly to body temperature to allow its correct positioning. Alternatively, the SMP can carry a degradable coating to allow its timed deployment. In another embodiment, the SMP implant body 60 can carry magnetic responsive elements for heating with an external inductive heating after implantation, or the polymer can carry radiosensitive elements for Joule heating thereof or chromophores for heating with light energy (endoluminal or by surface irradiation).

In any embodiment, the “structure” modulus can be equivalent to about 5 kPa to 2 MPa.

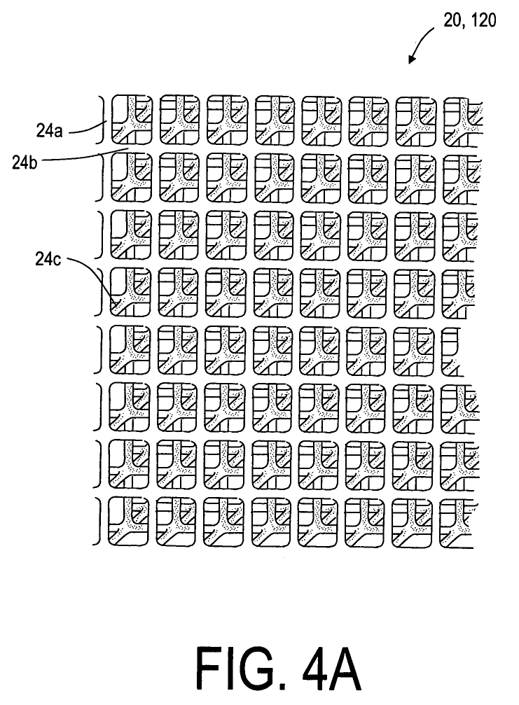

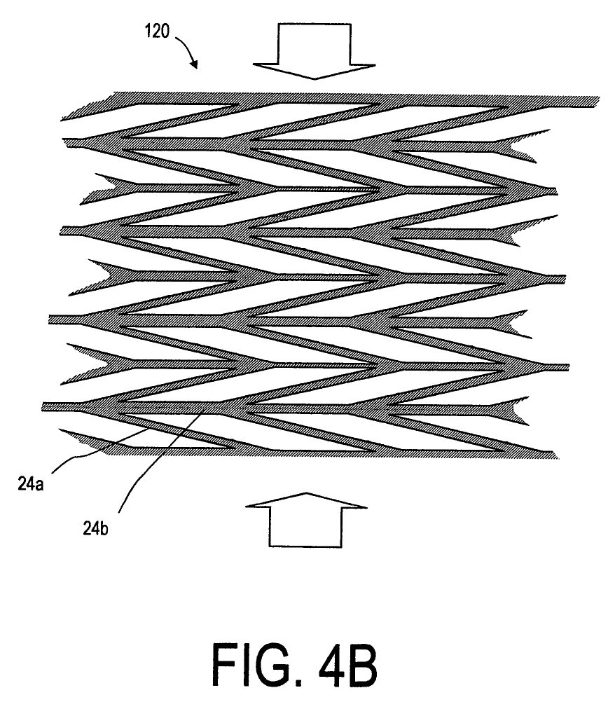

FIGS. 5-8 illustrate an alternative embodiment of SMP device for treating an aneurysm. FIG. 5 illustrates an exploded, sequential view of a method of making a neurovascular flow-excluding stent or implant 100A corresponding to the invention for treating an arteriovascular malformation (AVM). FIG. 6 illustrates a view of a portion of the assembled stent 100A that comprises a skeletal tubular stent body 102, for example, having shape memory alloy (SMA) struts 105 together with an open-cell elastomeric shape memory polymer (SMP) monolith or structure 120 that is adapted for coupling to the strut superstructure.

Of particular interest, the polymer element or structure 120 has a shape memory that cooperates with the shape memory of the SMA strut superstructure as in the assembled view of FIG. 6. As can be seen in FIG. 5, the skeletal stent body wall 102 is defined by struts 105 that circumscribe openings or cells 122. The SMP structure 120 is designed to be compactable to have an arrangement of struts 122′ that correspond to the stent superstructure.

FIG. 5 illustrates the novel microfabricated open-cell elastomeric element 120 in both its “memory” extended or expanded shape and its “temporary” non-extended of compacted shape de-mated from the struts 105. FIG. 6 illustrates the elastomeric element 120 in its “temporary” compacted shape mated to the strut superstructure. In a preferred embodiment, again as depicted in FIG. 4A, the open-cell elastomeric structure 120 is fabricated by soft lithography microfabrication means. An alternative method of making open-cell structure 120 is by a polymer foaming process. The open-cell polymer element 120, in its memory shape (its deployed configuration) is adapted for absorbing pulsatile forces caused by heart beats about the neck of an aneurysm. The open-cell elastomeric element 120 is further adapted to expand and partly occupy an AVM to limit or eliminate blood circulation within an aneurysm sac, which thereafter causes flow stagnation and natural thrombus formation to entirely occlude the aneurysm sac.

In FIG. 5, the open-cell elastomeric monolith 120 in its memory, repose shape is in a stress-free, non-skeletal state. FIG. 5 further illustrates that the elastomeric monolith 120 is capable being releasably maintained in a temporary, stressed, compacted and skeletal shape wherein the open-cell polymer is crushed, compacted and cut to have open cells 122′ that generally correspond to the open cells 122 in the skeletal strut superstructure. It should be noted that the term “cell” is being used in two different aspects in this disclosure, which are in common use (i) in describing stent superstructures having struts segments 105 that bound “cells”; and (ii) in describing polymer morphologies that include open “cell” foams and/or microfabricated open “cells” in the interior of a polymer monolith. The meaning of the term “cell” or “cells” as used herein will be apparent from the context, and generally the hyphenated term “open-cell” and “open-cell network” will be used when describing the open interior morphologies of the polymer monolith 120. The non-hyphenated terminology “open cell” will be used when describing the openings in the strut superstructure of a stent.

The use of an open-cell elastomeric SMP monolith 120 coupled to a strut superstructure allows for post-implant strain recovery that can resolve many of the vexing problems of occluding aneurysms in tortuous arteries and at treatment sites that carry many perforator vessels. In one aspect, the scope of the invention encompasses providing a skeletal stent superstructure with a SMP structure 120 carried about the exterior of selected struts. Of particular interest, the SMP component is designed to allow a temporary fixation of the monolith's shape, a selected strain recovery rate, and a selected capability of performing work during strain recovery to accomplish the objectives of the invention in controlling blood flow parameters (e.g., pulsatile forces, laminar flows, direction of circulation) about an aneurysm. In FIG. 5, the shape memory polymer structure is akin to a superelastic rubber composition wherein the polymer can be elevated in temperature to “rubbery” state and then deformed under a selected forces to overcome the materials resistance to deformation, and thereafter the temperature can be decreased to below a phase change temperature (e.g., glass transition temperature (T_{g}), melt temperature, (Tₘ), crystallization temperature or other phase change temperature) and the deformed shape can be fixed. Contemporaneously, the mechanical energy expended in deforming structure will be stored within the stressed monolith 120. When the temperature is elevated above the transition temperature (T_{g}, Tₘ or other phase transition), the elastomeric structure will exhibit strain recover and assume its original memory shape (see FIG. 5).

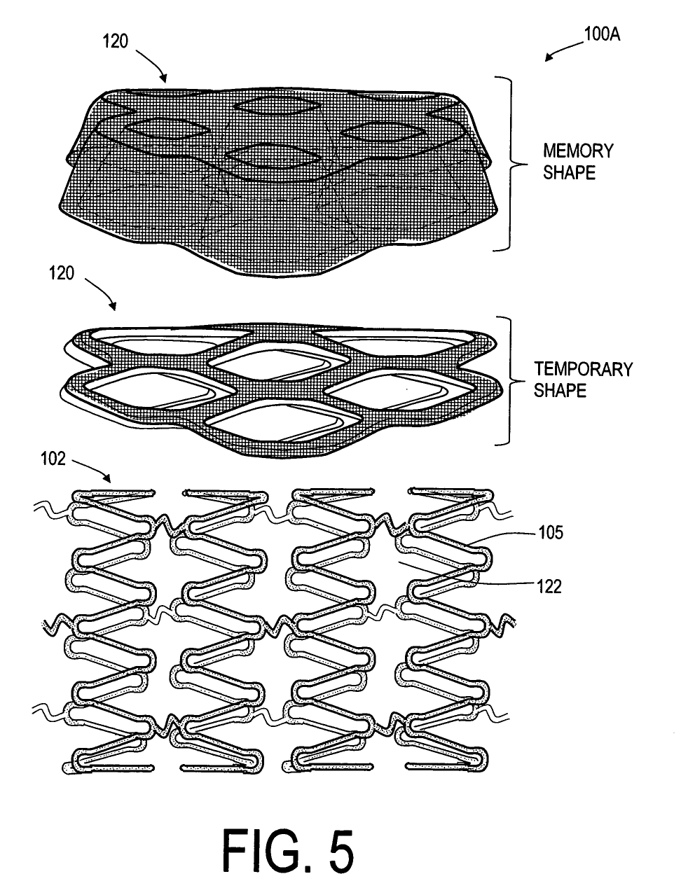

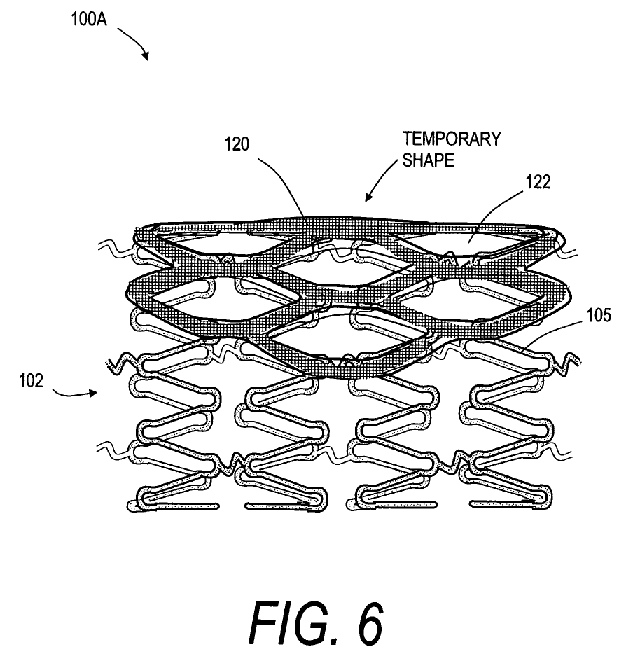

FIGS. 7-8 illustrate the stent in a pre-deployed position in a catheter 128 and deployed in a wide neck aneurysm, showing the lumen only for convenience.

The classes of SMPs described below will allow for large deformations, for example from about 20 percent to 500 percent or more. Further, the open-cell network of the SMP monolith 120 will allow its compaction to from depth D to a very thin layer depth indicated at D′ in FIG. 5. While the illustration of FIGS. 5 and 6 illustrate the thin compacted SMP element 120 has a width W′ that exceeds that of the struts 105, the SMP element also can be cut and formed to match the strut width or the struts in a certain portion of the skeletal superstructure can have an increased width W. The depth D of the elastomeric structure 120 in its memory shape can range from about 5 to 100 percent of the diameter of the strut superstructure.

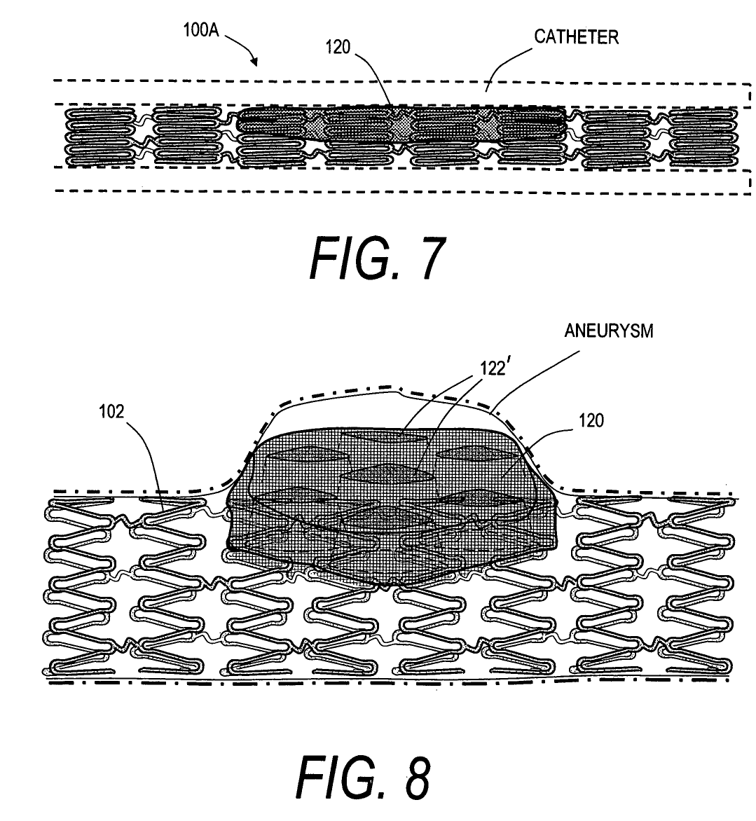

Of particular interest, the SMP element 120 can be fabricated to have a very low structure modulus (or structure stiffness)—so that its selected strain recovery rate and selected work capability is less than the recovery forces applied by the radially expanding SMA strut superstructure. Thus, as illustrated in FIG. 9, if the shape memory polymer body 120 in its compacted form is not exposed to the neck 144 of the aneurysm sac 145, but is pressed between the strut 105 and the wall of the parent vessel 148, that portion of the SMP monolith will simply remain in its compacted state. At any locations wherein the shape memory polymer body is free to extend outward from the strut into the AVM, the elastomer will extend to or towards its memory shape and thereby occupy a region of the AVM. The extended region 150 of the elastomeric monolith 120 in FIGS. 8 and 9 thus absorbs pulsatile forces of blood flow and excludes circulation from the AVM to cause its occlusion.

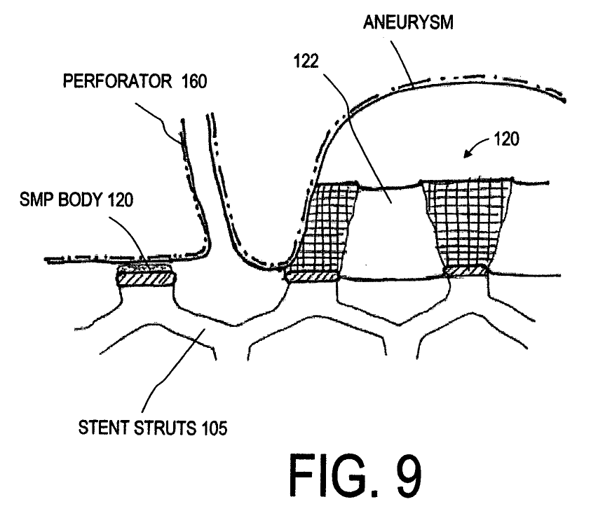

As can be seen understood from FIGS. 9 and 10, the elastomeric monolith 120 can also be configured as relatively thin layer of open-cell SMP polymeric film 155 that has a convex “memory” shape (FIG. 5A) that bulges outward from cells 122 of the strut superstructure 102. Such a memory shape that is convex and extends outwardly from the struts 105 will insure that the structure will not sag into the lumen of the parent vessel. The high deformation of the SMP film will allow the opening or cell 122′ in the film 155 to me expanded greatly and frozen in the temporary shape of FIG. 5B. The surface dimension E of cell 122′ in the memory shape can thus be substantially small as shown in FIG. 10.

FIG. 7 illustrates the stent 100A of FIGS. 1 and 2 with the assembly in its radially compressed configuration as when carried in a catheter sleeve 128 (phantom view). The catheter can be an over-the-wire system for introduction into, and navigation through, the lumen of the blood vessels. The stent 100A then is radially expandable from the shape of the non-expanded strut superstructure of FIG. 8 to the expanded superstructure as in FIG. 5.

A principal objective of the invention is to provide means to restrict or limit pulsatile blood flow within an intracranial aneurysm sac 145 while at the same time insuring that the stent superstructure 102 in the parent artery 148 has substantially large openings or cells 122 to limit the risk of any strut occluding a perforating side branch 160. Such perforating side branches 160 are shown in FIGS. 9 and 10, and often are located close to the neck 144 of an aneurysm 145. Such perforators 160 are very small in diameter, for example less than about 200-300 microns in cross section, and can provide the principal source of blood flow to critical sections of the brain. In the prior art, stents and sleeved stents that attempt to block the aneurysm neck 144 typically have a support structure that engages the parent vessel in a manner that will likely will block the local perforators 160. Such prior art stents thus may block blood flow through one or more perforators and cause a significant risk of ischemic stroke.

The stent corresponding to the invention addresses the issue of protecting perforators 160 with multiple innovations. First, the cross-section of struts 105 is substantially small (e.g., from 0.005″ to 0.050″) and the open cells 122 comprise a very large proportion of the stent body wall, with such cells 122 having open dimensions across a principal axis ranging form 0.5 mm to 2.0 mm or more. Second, the shape transformable elastomeric structure 120 can be provided in different selected dimensions and carried on different stent body portions, thus allowing selection of the particular dimension or profile of the memory shape of the polymer structure 120. Third, in several embodiments, the SMP structure 120 is has an open-cell structure of a selected radial thickness will immediately reduce the velocity of blood flow, or eliminate circulation in the sac altogether, to thereafter allow blood to clot naturally within the open-cells of the polymer to block the neck of the aneurysm. Fourth, as described above, the shape transformable structure 120 also in of an ultra low modulus polymer that is adapted to only expand into an aneurysm neck 144 and not between a strut and an engaged vessel wall thus preventing expansion of the polymer into a perforator 160. Fifth, in several embodiments, the open-cell SMP structure 120 can be of a bioerodible polymer that rapidly erodes to further insure that the structure does not block any perforator 160. Sixth, in all embodiments, the SMP structure 120 is stress-free in its expanded shape to insure that no unwanted expansion forces are applied to the aneurysm sac 145 as would be typical in packing the sac within embolic coils.

As can be seen in FIGS. 9 and 10, a stent 100A can be adapted to treat either berry-type aneurysms or wide-neck aneurysms. The shape-transformable elastomeric structure 120 will substantially limit blood circulation and in particular (i) will limit pulsatile effects caused by the heart's rhythmic beating, and (ii) will limit the velocity and laminar flows (or increase the turbulence) of any remaining circulation in and about the AVM that applying distending forces to the aneurysm sac. In the absence of such pulsatile effects and laminar flows, stagnant blood can be allowed to form thrombus the thereafter cause the wall of the AVM to collapse. The stent of the invention is particularly adapted for use in narrow and highly tortuous vasculature characterized by having numerous perforators 160.

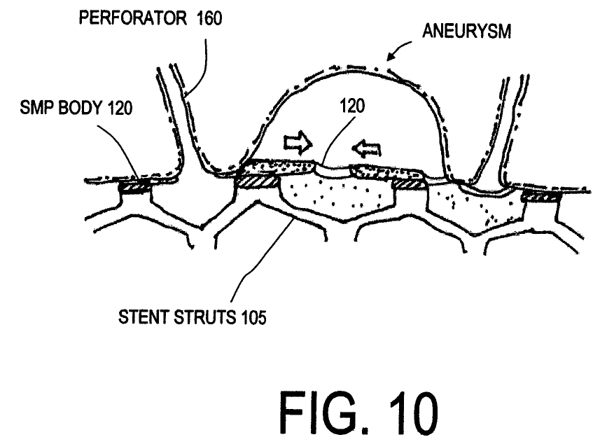

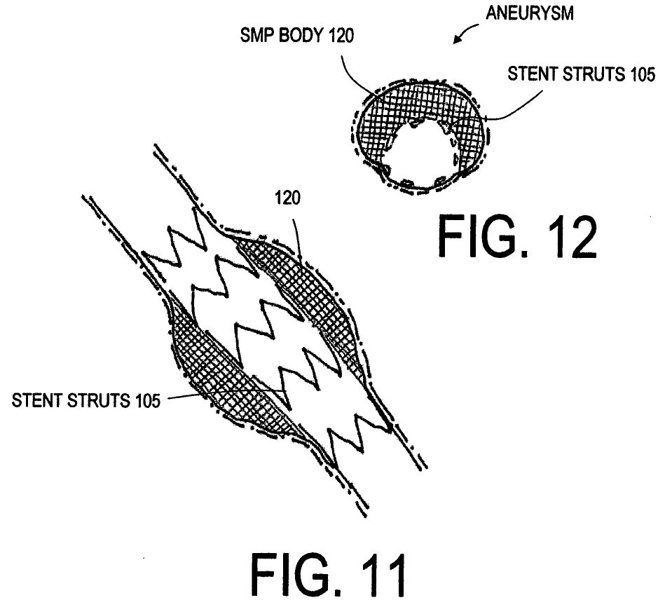

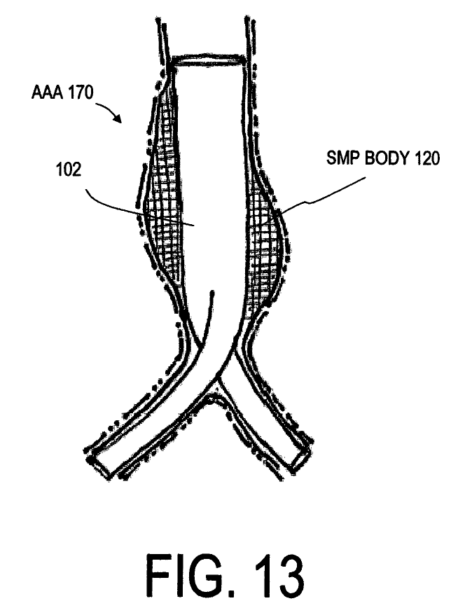

It can be understood that the strut superstructure 102 as in FIGS. 11-12 can carry a shape memory polymer structure 120 that extends from 90 to 360 degrees around a portion of the stent for treating fusiform aneurysms or large aneurysms. A stent corresponding to the invention also can be fabricated is large dimensions, as in FIG. 13. In this embodiment, the stent has a superelastic SMA strut superstructure 102 with 100 percent surface coverage with a thin or thick SMP structure 120 for abdominal aortic aneurysms (AAA). Such an AAA stent can have a linear form or a bifurcated form as in FIG. 13. Means for coupling the stent, or stent graft, to the vessel wall can be included and are well known in the art. In the schematic views of FIG. 13, it can be seen that a low modulus open-cell polymer structure 120 is adapted to expand, fill and occlude that aortic aneurysm 170. The expandable structure 120 also can serve to engage the vessel wall to maintain the stent in the desired location.

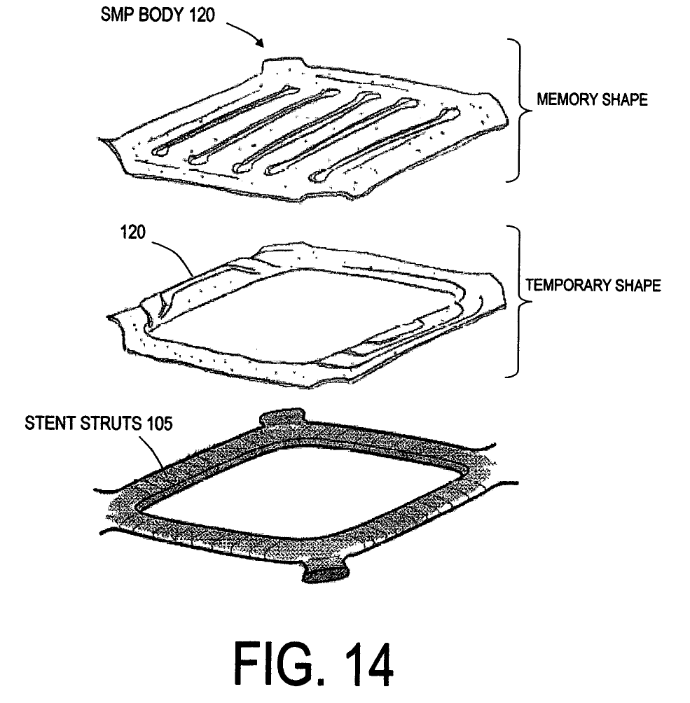

In another preferred embodiment, the strut superstructure 102 of a stent as shown in FIG. 14 can carry a thin shape-transformable polymer structure 220 that is selectively deployable across one or more stent open cells. The polymer can be deployable in response to a selected stimulus. The stimulus can be heat applied by light energy, Rf energy, magnetic energy or the like. The polymer can be a heat shrink polymer film as is known in the art or a shape memory polymer as described below.

In the exploded view of FIG. 14, it can be seen that the polymer structure 220 is a “cut” thin film element. FIG. 14 illustrates that the memory shape can be deformed to essentially conform to the shape and dimensions of the struts 105 to which the polymer structure is bonded for introduction into the targeted location. This type of shape-transformable polymer can be very useful for treating aneurysm where perforators are present as illustrated above in FIGS. 9 and 10. The scope of the invention encompasses varied forms of thin structures that can be deformed and carried by the struts of a stent and that can be transformed in shape to extend between the struts to obstruct blood flow through the struts into an AVM. The scope of the invention includes knit, braided and woven structures of polymer microfilaments that can be stimulated to shrink and thus extend across the opening between struts.

It should be appreciated that the scope of the invention includes using gradients in the structure modulus, or stiffness, of the SMP structure 120 to allow the neck of the aneurysm or vessel wall to be engaged with a higher modulus portion while the distended aneurysm wall is engage by a lesser modulus portion.

The stent 100A as described above has shape memory alloy struts and is a self-expanding stent. Vascular stents similar to that of FIGS. 5-8 also can be in the class of balloon-expandable stents that are made of a deformable material. Such a balloon-expandable stent is typically made of a stainless steel tube that is laser cut to form the interconnected struts of the stent body wall. Thus, the stent in a first smaller cross-section or non-expanded configuration for introduction through vasculature after being deformed or crimped about a balloon catheter. The stent is capable of the second, expanded configuration, upon the application of radially, outwardly directed forces by the balloon. A representative balloon-expandable stent is described in U.S. Pat. Nos. 4,776,337 and 4,733,665 to Palmaz. The shape memory polymer structure 120 of the invention also can be incorporated into such balloon-expandable stents. Stents fabricated of a metal strut superstructure, whether pressure-expandable or self-expanding, vary considerably in their geometric forms. Any such geometric form may be suitable for the present invention. Thus, the exemplary stent can have a strut superstructure of a biocompatible material such as a deformable metal (stainless steel, tungsten, titanium, gold, platinum and tantalum and alloys thereof) or shape-memory alloys.

The shape memory polymer structure 120 of the invention also can be incorporated into any type of polymer stent known in the art, e.g., foldable types, self-expanding types, thermoset type and the like. A polymer expandable stent is disclosed in U.S. Pat. No. 5,163,952 to Froix. A polymer stent body also can be shape memory polymer.

In order to better describe elastomeric structure 120 of FIGS. 1 and 2 that is fabricated of a shape memory polymer, it is first useful to provide background on such SMPs. SMPs demonstrate the phenomena of shape memory based on fabricating a segregated linear block co-polymer, typically of a hard segment and a soft segment. The shape memory polymer generally is characterized as defining phases that result from glass transition temperatures (T_{g}) in the hard and soft segments or other types of phase change. The hard segment of SMP typically is crystalline with a defined melting point, and the soft segment is typically amorphous, with another defined transition temperature. In some embodiments, these characteristics may be reversed together with the segment's glass transition temperatures. In one embodiment, the SMPs that are suitable for the implant are a subset of shape memory polymer materials that comprises an open-cell foam polymer. Such open-cell foam SMPs also for compaction of the structure.

Referring to FIG. 5, the SMP structure 120 can be fabricated to the indicated memory shape. In such an embodiment, when the SMP material is elevated in temperature above the melting point or glass transition temperature of the hard segment, the material is then formed into its memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed. The temporary shape can be a highly compacted shape for pre-deployment storage.

The original memory shape is recovered by heating the material above the melting point or glass transition temperature T_{g} of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. for a typical embodiment. Alternatively, a higher transition temperature can be selected and remote source can be used to elevate the temperature and expand the SMP structure to its memory shape (i.e., inductive heating or light energy absorption).

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, permeability and index of refraction. Examples of polymers that have been utilized in hard and soft segments of SMPs include polyurethanes, polynorborenes, styrene-butadiene co-polymers, cross-linked polyethylenes, cross-linked polycyclooctenes, polyethers, polyacrylates, polyamides, polysiloxanes, polyether amides, polyether esters, and urethane-butadiene co-polymers and others identified in the following patents and publications: U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al. (all of which are incorporated herein by reference); Mather, Strain Recovery in POSS Hybrid Thermoplastics, Polymer 2000, 41(1), 528; Mather et al., Shape Memory and Nanostructure in Poly(Norbonyl-POSS) Copolymers, Polym. Int. 49, 453-57 (2000); Lui et al., Thermomechanical Characterization of a Tailored Series of Shape Memory Polymers, J. App. Med. Plastics, Fall 2002; Gorden, Applications of Shape Memory Polyurethanes, Proceedings of the First International Conference on Shape Memory and Superelastic Technologies, SMST International Committee, pp. 120-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties of shape memory polymers of polyurethane series and their applications, J. Physique IV (Colloque C1) 6:377-84 (1996)) (all of the cited literature incorporated herein by this reference). The above background materials, in general, describe SMP in a non-open cell solid form. The similar set of polymers can be foamed, or can be microfabricated with an open cell structure for use in the invention.

Shape memory polymers foams that fall within the scope of the invention typically are polyurethane-based thermoplastics that can be engineered with a wide range of glass transition temperatures. These SMP foams possess several potential advantages for the invention, for example: very large shape recovery strains are achievable, e.g., a substantially large reversible reduction of the Young's Modulus in the material's rubbery state; the material's ability to undergo reversible inelastic strains of greater than 10%, and preferably greater that 20% and still more preferably greater that about 100; shape recovery can be designed at a selected temperature between about 30° C. and 60° C. which will be useful for the treatment system, and injection molding is possible thus allowing complex shapes. These polymers also demonstrate unique properties in terms of capacity to alter the material's water or fluid permeability and thermal expansivity. However, the material's reversible inelastic strain capabilities leads to its most important property—the shape memory effect. If the polymer is strained into a new shape at a high temperature (above the glass transition temperature T_{g}) and then cooled it becomes fixed into the new temporary shape. The initial memory shape can be recovered by reheating the foam above its T_{g}.

Of particular interest, as illustrated in FIG. 4A, a preferred elastomeric structure 120 is be microfabricated using soft lithography techniques to provide an open-cell networked interior which will absorb, dampen blood flow velocity and result in clotting. Preferably, the structure 120 as in FIG. 4A is molded in layers assembled by one or more soft lithographic techniques. An open-cell structure can be microfabricated of a resilient polymer (e.g., silicone) by several different techniques-all collectively known as soft lithography. For example, microtransfer molding is used wherein a transparent, elastomeric polydimethylsiloxane (PDMS) stamp has patterned relief on its surface to generate features in the polymer. The PDMS stamp is filled with a prepolymer or ceramic precursor and placed on a substrate. The material is cured and the stamp is removed. The technique generates features as small as 250 nm and is able to generate multilayer systems that can be used to fabricate the stent as well as lumen 120. Replica molding is a similar process wherein a PDMS stamp is cast against a conventionally patterned master. A polyurethane or other polymer is then molded against the secondary PDMS master. In this way, multiple copies can be made without damaging the original master. The technique can replicate features as small as 30 nm. Another process is known as micromolding in capillaries (MIMIC) wherein continuous channels are formed when a PDMS stamp is brought into conformal contact with a solid substrate. Then, capillary action fills the channels with a polymer precursor. The polymer is cured and the stamp is removed. MIMIC can generate features down to 1 μm in size. Solvent-assisted microcontact molding (SAMIM) is also known wherein a small amount of solvent is spread on a patterned PDMS stamp and the stamp is placed on a polymer, such as photoresist. The solvent swells the polymer and causes it to expand to fill the surface relief of the stamp. Features as small as 60 nm have been produced. A polymeric microstructure as in a stent can be entirely of a “Lincoln-log” type of assembly similar to that shown in Xia and Whitesides, Annu. Rev. Mater. Sci. 1998 28:153-84 at p. 170, FIG. 7d (the Xia and Whitesides article incorporated herein by reference).

Any polymer structure 120 (FIGS. 13-14) also can have a surface modification to enhance thrombus formation, or can carry pharmacological agents to induce clotting.

Those skilled in the art will appreciate that the exemplary embodiments and descriptions thereof are merely illustrative of the invention as a whole. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention.

1. A stent for treating an arteriovascular malformation (AVM) comprising a skeletal support structure for expanding in a blood vessel and a shape-transformable polymer structure coupled to surface portions of the support structure.

2. A stent as in claim 1 wherein the shape-transformable polymer structure has a first shape that is skeletal and a second shape that is non-skeletal.

3. A stent as in claim 2 wherein the second shape is configured to alter blood flow parameters to treat an AVM.

4. A stent as in claim 2 wherein the second shape is configured to extend at least in part outwardly from the skeletal support structure.

5. A stent as in claim 2 wherein the second shape is configured to extend within openings of the skeletal support structure.

6. A stent as in claim 1 wherein the shape-transformable polymer structure includes a shape memory polymer.

7. A stent as in claim 2 wherein the shape-transformable polymer structure is a rofabricated open-cell material.

8. A stent as in claim 2 wherein the shape-transformable polymer structure is a foam.

9. A stent as in claim 1 wherein the shape-transformable polymer structure includes a t shrink polymer.

10. A stent as in claim 1 wherein the shape-transformable polymer structure is at least of knit, woven or braided.

11. A stent as in claim 2 wherein the skeletal support structure is at least one of a metal or a polymer.

12. An implant body for treating vasculature including a shape memory polymer capable of a first temporary compacted shape for endovascular introduction and a second memory expanded shape for altering blood flow parameters in a targeted region of the vasculature.

13. A stent as in claim 2 wherein the shape memory polymer structure includes at least one of a microfabricated open cell portion and an open-cell foam portion.

14. An implant body as in claim 12 wherein the shape memory polymer is coupled to struts of an expandable stent.

15. An implant body as in claim 12 wherein the shape memory polymer has an elongated configuration for occluding a blood vessel.

16. An implant body as in claim 12 wherein the shape memory polymer comprises a constraint for constraining an interior portion of the implant body.

17. An implant body as in claim 12 wherein the shape memory polymer is at least oen of bioerodible and bioabsorbable.

18. A method of treating an arteriovascular malformation (AVM) comprising introducing a stent in a collapsed shape into a blood vessel, expanding the stent in or proximate to an AVM, and altering the shape of a shape-transformable polymer structure coupled to surface portions of the stent to alter blood flow parameters in or proximate to the AVM.

19. A method of treating an arteriovascular malformation (AVM) as in claim 18 wherein the shape-transformable polymer structure extends radially outward from surface portions of the stent.

20. A method of treating an arteriovascular malformation (AVM) as in claim 18 wherein the shape-transformable polymer structure extends laterally within openings between struts of the stent.
